(19) United States
(12) Patent Application Publication
(10) Pub. No.: US 2013/0259821 A1
(43) Pub. Date: Oct. 3, 2013

(54) FORMULATION COMPRISING POLYSILOXANES HAVING NITROGEN-CONTAINING GROUPS

(71) Applicants: Frauke Henning, Essen (DE); Michael Ferenz, Essen (DE); Sascha Herrwerth, Essen (DE); Christian Hartung, Essen (DE)

(72) Inventors: Frauke Henning, Essen (DE); Michael Ferenz, Essen (DE); Sascha Herrwerth, Essen (DE); Christian Hartung, Essen (DE)

(73) Assignee: EVONIK GOLDSCHMIDT GMBH, Essen (DE)

(21) Appl. No.: 13/992,311

(22) PCT Filed: Nov. 15, 2011
(86) PCT No.: PCT/EP11/70091

§ 371 Date: Jun. 7, 2013

(30) Foreign Application Priority Data
Dec. 9, 2010 (DE) 10 2010 062 676.7

(51) Int. Cl. A61K 8/898; A61Q 5/06; A61Q 19/00; A61Q 5/12

(52) U.S. Cl. CPC A61K 8/898; A61Q 5/12; A61Q 5/06; A61Q 19/00
USPC 424/70.122; 528/34; 514/772.3

(57) ABSTRACT

The invention relates to the use of multiamino-functional polysiloxanes in formulations and to the formulations comprising these polysiloxanes.

### FIELD OF THE INVENTION

The field of the invention relates to cosmetic preparations comprising polysiloxanes which are laterally modified with amino functions and at least one further polar functional group in defined ratios, and to the use of these preparations in cosmetic formulations for the care of skin and keratin fibres.

### PRIOR ART

Amino-functional siloxanes are widely used as conditioners for textiles, additives in shampoos and haircare products and hydrophobicizing agents. A multiplicity of structural variations of this substance group is described in the prior art and is accessible via various production routes. In this connection, purely linear polydimethylsiloxanes terminally modified with amino groups are not variable in their degree of modification. This is disadvantageous since both the number of amino groups, and also the type of amino groups have considerable influence on the aminopolysiloxane's substantivity, i.e. on the ability to bind to carriers such as, for example, keratin substances. The total nitrogen content of an aminosiloxane is an important parameter because it correlates directly with its substantivity.

ABn multiblock copolymers are described for extending the polymer chain without reducing the amino functionalities. However, as chain length increases, linear copolymers become very viscous and hence difficult to handle. U.S. Pat. No. 5,807,956 and U.S. Pat. No. 5,981,681 teach non-hydrolysable block copolymers of the (AB)nA type with alternating units consisting of polysiloxane and amino-polyalkylene oxide. Here, α,ω-dihydrogenpolydimethylsiloxanes are linked by means of noble-metal-catalyzed hydrosilylation to olefins carrying epoxide groups in an SiC manner, and the epoxy-terminated siloxanes produced in this way are reacted with amino-terminated polyalkylene oxides. Alternatively, α,ω-dihydrogenpolydimethylsiloxanes are linked to epoxy-terminated allyl polyethers by hydrosilylation, and the epoxy-functionalized siloxanes obtained in this way are subsequently reacted with diamines.

Polysiloxanes with high degrees of modification coupled with a chain length which can be varied irrespective of the nitrogen content are obtainable by lateral functionalization of a polysiloxane with organic substituents containing amino groups.

The prior art discloses a multitude of references relating to laterally modified aminosiloxanes which are currently used in large amounts in cosmetic formulations. For example, Momentive SF 1708 (INCI: Amodimethicone, Momentive), DC 2-8566 (INCI: Amodimethicone, Dow Corning) and KF-865 (INCI: Aminopropyl Dimethicone, Shin Etsu) are commercially available.

The preparation of laterally modified aminosiloxanes can take place under base catalysis or acid catalysis. Preparation by base-catalyzed equilibration, as explained, for example, in EP 1972330 A2 in paragraphs 0154 and 0155, can lead, depending on the starting materials used, either to terminally dihydroxy-functional, laterally amino-modified polysiloxanes, or to laterally amino-modified polysiloxanes, the chain ends of which are end-capped with trimethylsilyl groups. Such end-capped polysiloxanes when compared with their structural analogue provided with condensable groups, such as, for example SiOH or SiOR groups (R=for example methyl and ethyl radical), not only have a better storage stability in the absence of a solvent, but also prevent gel-like precipitations and accretions during the handling of aqueous emulsions of such polysiloxanes.

According to the prior art, as described, for example, in U.S. Pat. No. 7,238,768 B2, the acid-catalyzed condensation polymerization leads to amino-modified polysiloxanes with hydroxyl groups or alkoxy groups at the chain ends. Although the process is advantageous compared with base-catalyzed equilibrations on account of lower reaction temperatures and shorter reaction times, the more cost-effective production process nevertheless brings about the shortcoming of reduced stability of these non-end-capped siloxanes on account of the lack of trimethylsilyl end groups.

For example, U.S. Pat. No. 6,171,515 B1 describes end-capped and also dialkoxy-functional aminopolysiloxanes which, in a synthesis step downstream of the siloxane polymerization, undergo a functionalization of the primary and secondary amino groups with epoxy-functional monomers, such as, for example, glycidol. A similar functionalization of aminosiloxanes with alkylene oxides is described in DE 69003009 T2. Further functionalizations of amino-functional polysiloxanes with glycerol carbonate or gluconolactone are described in EP 192330 A2 or in J. Phys. Chem. B 2010, 114, 6872-6877.

The derivatization of the amino function has a significant influence on the substantivity of the nitrogen-containing polysiloxanes on skin or keratin fibres. In particular, the sensory properties of the cosmetic formulations are dependent on the type and amount which is deposited on skin or hair during the application of the nitrogen-containing polysiloxanes used.

For increasing the substantivity, JP 2002-167437 A describes polysiloxanes laterally functionalized with guanidino radicals, which are prepared by reacting the corresponding aminopolysiloxanes with cyanamide. DE 102005004704 A1 describes the condensation copolymerization of a dihydroxy-functional polydimethylsiloxane with a guanidino-group-containing silane and an amino-group-containing silane. Although a functionalization of the polysiloxane with nitrogen-containing groups that differ in type and amount is possible in this way, DE 102005004704 does not disclose any route to end-capped multiamino-functional polysiloxanes.

A disadvantage of all of the aminopolysiloxanes described in the prior art is, inter alia, their viscosity reduction of the formulations containing them. It is therefore desirable from a formulation point of view to use the lowest possible amounts of amino-functional siloxanes without having to accept significant losses in performance.

It is an object of the present invention to provide cosmetic care active ingredients with good application properties.

### DESCRIPTION OF THE INVENTION

Surprisingly, it has been found that nitrogen-containing polysiloxanes of the general formula 1 are outstanding cosmetic care active ingredients.

The present invention thus provides the use of polysiloxanes as described in claim 1 as care active ingredient in dermatological, cosmetic and pharmaceutical applications and also corresponding formulations comprising the nitrogen-containing polysiloxanes of the general formula 1.

It is one advantage that the nitrogen-containing polysiloxanes of the general formula 1 bring about an improved conditioning of skin and hair than aminopolysiloxanes known hitherto.

Another advantage of the present invention is that the nitrogen-containing polysiloxanes of the general formula 1 have very good substantivity.

On account of the aforementioned advantages, a higher effectiveness is obtained based on a good effect coupled with a reduced use amount in the formulation.

A reduction in the use amount of amino-functional polysiloxanes has the advantage that the formulation and incorporation processes are simplified.

For example, as a result of the reduced amount of amino-functional active ingredients for the same or better conditioning of skin and hair, smaller amounts of thickener are required in a cosmetic surface-active formulation since amino-functional siloxanes generally have a diluting effect in surface-active formulations (for example in shampoos or shower gels).

This hand-in-hand reduced use of active ingredient and thickener leads to a preservation of resources.

It is yet a further advantage of the present invention that the polysiloxanes used according to the invention are precisely defined polymers in terms of structure, whose nitrogen content, type and amount of amino groups and chain length thereof can be adjusted independently of one another in a variable manner via the formulation, such that a constant composition and a reproducible quality of the polymers containing the amino groups is given with regard to the respective application.

It is also an advantage of the present invention that the nitrogen-containing polysiloxanes used are able to improve both properties such as combability, softness, volume, shapeability, handleability, de-tangleability of undamaged and damaged hair, and also impart a nice shine to the hair.

The present invention provides the use of at least one polysiloxane of the general formula 1

MₐD_{b}D^{A}_{c}D^{B}_{d}D^{c}ₑT_{f}Q_{g} (formula 1)

### M=[R²R¹₂SiO_{1/2}]

### D=[R¹₂SiO_{2/2}]

### D^{A}=[R¹Si(R⁷NHR³)O_{2/2}]

### D^{B}=[R¹SiR⁴O_{2/2}]

### D^{C}=[R¹SiR⁵O_{2/2}]

### =T=[R¹SiO_{3/2}]

### Q=[SiO_{4/2}],

where
R¹ independently of the others, is identical or different linear or branched, saturated or unsaturated hydrocarbon radicals having 1 to 30 carbon atoms or else aromatic hydrocarbon radicals having 6 to 30 carbon atoms, preferably methyl or phenyl, in particular methyl;
R² independently of the others is the same as R¹, an alkoxy radical or a hydroxy group, preferably R¹, in particular methyl;
R³ independently of the others, is hydrogen or a hydrocarbon radical substituted with nitrogen atoms, for example an aminoethyl radical, in particular hydrogen;
R⁴ independently of the others, is identical or different, linear or branched, saturated or olefinically unsaturated hydrocarbon radicals having 8 to 30 carbon atoms, for example decyl, dodecyl, tetradecyl, hexadecyl, octadecyl, in particular hexadecyl and octadecyl;
R⁵ independently of the others, is identical or different, linear or branched, saturated or unsaturated polar hydroxy-substituted amide radicals having 1 to 30 carbon atoms and/or hydroxy-substituted carbamate radicals having 1 to 30 carbon atoms and/or ethoxylated amine radicals having 1 to 30 carbon atoms and/or guanidine radicals or alkylenylguanidine radicals having 1 to 30 carbon atoms, preferably selected from the group of the substituents of the formula 1a to 1 h, in particular 1e and 1f,

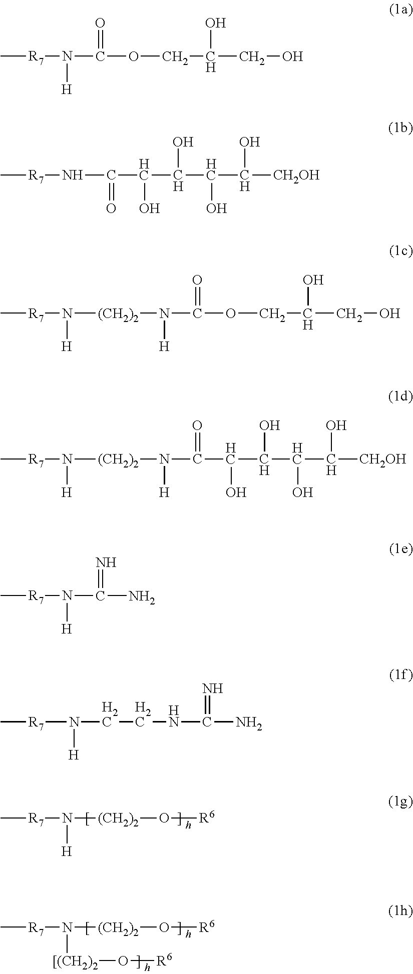

R⁶ is hydrogen, a hydrocarbon radical, an acyl radical, a carboxylate radical or a carbamate or carbonate radical, in particular hydrogen and CH₃—C(O);
R⁷ independently of the others, is identical or different linear or branched, saturated or unsaturated, divalent hydrocarbon radicals, preferably —(CH₂)₃—
a=2 to 20, preferably 2 to 10, in particular 2,

b=10 to 5000, preferably 20 to 2000, in particular 20 to 1000,

c=1 to 500, preferably 1 to 100, in particular 1 to 30,
d=0 to 500, preferably 0 to 100, in particular 0 to 30,
e=0 to 500, preferably 1 to 100, in particular 1 to 30,
f=0 to 20, preferably 0 to 10, in particular 0,
g=0 to 20, preferably 0 to 10, in particular 0,
h=0 to 20, preferably 1 to 10, in particular 1-2,
or ionic adducts thereof with protic reactants H⁺A⁻,
with the proviso that at least 50%, preferably at least 70% of the radicals R²═R¹ and that at least one of the indices d and e≠0 and that if d=0, e≠0, and if e=0, d≠0, preferably c≧1, e≧1 and c>0.5*e, in particular c≧1, e≧1 and c≧e,
as care active ingredient, in cosmetic, dermatological or pharmaceutical formulations.

A process for the preparation of the polysiloxanes used according to the invention or present in the formulations according to the invention is based on the use of the compounds specified below by way of example, where

a) terminally hydroxy-functional linear or branched polysiloxanes and mixtures thereof with dimethyldialkoxysilanes or methyltrialkoxysilanes, preferably linear terminally dihydroxy-functional polysiloxanes,
b) hexamethyldisilazane or disilazanes substituted with various carbon radicals, such as, for example, divinyltetramethyldisilazane, preferably hexamethyldisilazane and
c) 3-aminopropylmethyldialkoxysilanes, N-(2-aminoethyl)-3-aminopropylmethyl-dialkoxysilanes or further functional dialkoxysilanes which contain linear or branched, saturated or unsaturated, hydrocarbon radicals which are substituted with hydroxy-substituted amides and/or hydroxy-substituted carbamate structures and/or ethoxylated amines and/or guanidine or alkylenylguanidine structures, or are selected from the group of the substances of the formulae 2a-i:

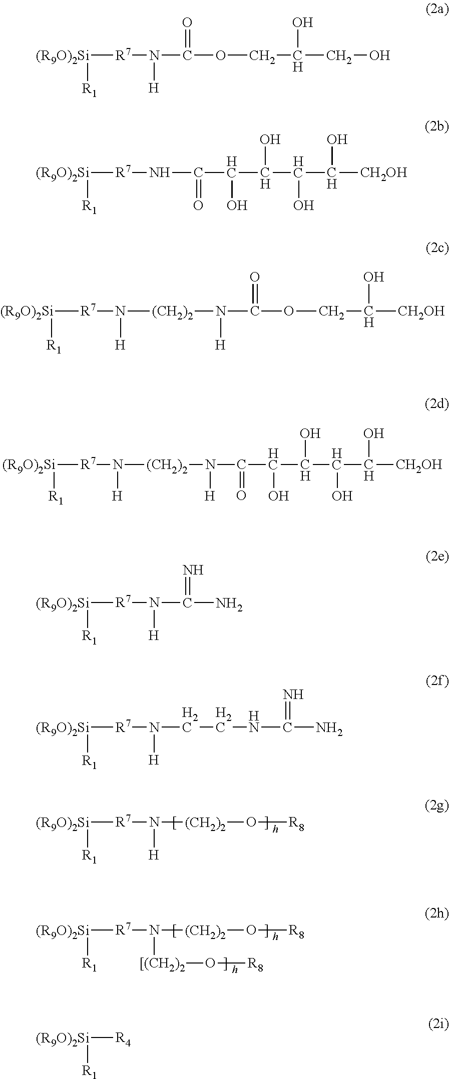

where R¹, R⁴, R⁷ and h have the aforementioned meaning of the formula 1, R⁸ is a hydrogen atom, a methyl or a carboxyl group, preferably H or acetyl, and R⁹ is an alkyl or acyl radical, in particular methyl, ethyl or acetyl, are reacted together.

The silanes are expediently used as monomer. Provided it is advantageous for the subsequent application, the silanes can be pre-condensed to give oligomers under hydrolytically acidic conditions before the polymer build-up is initiated by adding the dihydroxy-functional polysiloxanes.

It may be advantageous to use the silazane used not in stoichiometric amounts, but in a slight excess. The dialkoxysilanes used are prepared using synthesis methods known in the prior art. For example, the guanidation of amino-functional silanes takes place analogously to the guanidation of aminosiloxanes described in JP 2002 167437. The reaction of amino-functional siloxanes with glycerol carbonate or gluconolactone takes place in accordance with EP 1 972 330 A1 and J. Phys. Chem. B 2010, Vol. 114, pp. 6872-6877. The catalysts used for the hydrolysis and condensation reaction are carboxylic acids such as, for example, acetic acid, propionic acid, isononanoic acid or oleic acid. The reaction can be carried out with the addition of small amounts of water to increase the rate of the hydrolysis; often, adequate moisture is present in the reagents used in undried form. The reaction can be carried out in the absence of a solvent or in the presence of solvents, such as, for example, in aliphatic and aromatic, protic and aprotic solvents, glycols, ethers, fatty alcohol alkoxylates, mono-, di- and triglycerides or oils of synthetic and natural origin. The use of solvents is advantageous for example if the chain lengths of the desired structures bring about high viscosities. The solvent can be metered in before, during or after the reaction. The reaction can be carried out at temperatures in the range from room temperature to 150° C., preferably at 50-100° C. The alcohols released in the hydrolysis are distilled off in vacuo during or after the reaction. Optionally, a neutralization step and a filtration step can take place. The different monomer units of the siloxane chains given in the formulae can be constructed block-wise among one another with any desired number of blocks and any desired sequence, or be subject to statistical distribution. Ionic adducts of the amino-functional siloxanes used according to the invention or present in the formulations according to the invention with protic reactants H⁺A⁻ are in the form —NH₃⁺A⁻. The anions A⁻ are identical or different counterions to the positive charges on the protonated, primary amino groups, selected from inorganic or organic anions of the acids H⁺A⁻, and also derivatives thereof. Preferred anions are chloride, sulphate or hydrogensulphates, carbonate or hydrogencarbonate, phosphate or hydrogenphosphates, acetate or homologous carboxylates with linear or branched, saturated or olefinically unsaturated alkyl chains, aromatic carboxylates, carboxylates formed from amino acids, citrates, malonates, fumarates, maleates, substituted and unsubstituted succinates and carboxylates formed from L-hydroxycarboxylic acids, such as, for example, lactate. The aminosiloxanes and their ionic adducts can naturally be present in dissociation equilibria depending on the stability of the adduct formed. The indices used in the formulae are to be regarded as statistical average values.

Here, the term “care active ingredient” is understood as meaning a substance which fulfils the purpose of retaining an article in its original form or of masking, reducing or avoiding the effects of external influences (for example time, light, temperature, pressure, soiling, chemical reaction with other reactive compounds that come into contact with the article) such as, for example, ageing, soiling, material fatigue, bleaching, or of even improving desired positive properties of the article. For the last point, mention may be made for example of improved hair shine or a greater elasticity of the article under consideration.

According to the invention, preference is given to using polysiloxanes for which R⁵ in formula 1 contains at least one substituent selected from the formulae 1a to 1f, in particular 1e and 1f, with the proviso that the index c in formula 1 is greater than the index e.

Furthermore, preference is given to using polysiloxanes for which R¹ and R², independently of one another, are identical or different and are an alkyl radical having 1 to 4 carbon atoms, in particular R¹═R²=methyl.

According to the invention, water-soluble or water-insoluble polysiloxanes according to formula 1 can be used. Depending on the formulation to be produced (cloudy or clear formulations), it is known to the person skilled in the art whether water-soluble or insoluble polysiloxanes should be used to prepare the formulation. Within the context of the present invention, the term “water-insoluble” is defined as a solubility of less than 0.01 percent by weight in aqueous solution at 20° C. and 1 bar pressure. Within the context of the present invention, the term “water-soluble” is defined as a solubility of more than or equal to 0.01 percent by weight in aqueous solution at 20° C. and 1 bar pressure.

The use according to the invention of the polysiloxanes according to formula 1 as care active ingredient preferably takes place in surfactant-containing, in particular in surfactant-containing aqueous, formulations, the term “aqueous” in this context being understood as meaning formulations which have at least 40% by weight, in particular at least 60% by weight, very particularly at least 75% by weight, of water, based on the total formulation.

A use preferred according to the invention as care active ingredient is the use as conditioner, in particular as conditioner for skin and hair, preferably for hair. Consequently, the polysiloxanes according to formula 1 are preferably used in hair treatment compositions and hair after-treatment compositions. In this connection, the use according to the invention is preferably carried out in particular in hair treatment compositions and hair after-treatment compositions for rinsing out or for leaving in the hair, for example in shampoos with or without a marked conditioning effect, 2-in-1 shampoos, rinses, hair treatments, hair masks, hair styling assistants, styling compositions, blow-drying lotions, hair setting compositions, perming compositions, hair smoothing compositions and compositions for colouring the hair.

For the use according to the invention, the polysiloxanes according to formula 1 are advantageously used in the formulations in a concentration of from 0.01 to 20 mass percent, preferably 0.1 to 8 mass percent, particularly preferably from 0.2 to 4 mass percent, very particularly preferably from 0.2 to 1.0, in particular up to 0.7, mass percent, based on the total formulation.

It has been found that the polysiloxanes according to formula 1 can be used advantageously for dispersing particles, in particular in cosmetic, dermatological, or pharmaceutical formulations. Consequently, the polysiloxanes according to formula 1 according to the invention can additionally be used as dispersion auxiliaries of particles, in particular of metal oxides, in particular of nanoparticulate TiO₂, which can be hydrophobically or hydrophilically modified, of coloured pigments, such as, for example, FeₓO_{y} (iron oxide), mica, ZnO, titanium dioxide or manganese dioxide, where the formulations are preferably selected from the list consisting of sunscreen compositions, decorative cosmetic formulations, such as, for example, lipsticks, make-up, mascara, foundations or blemish creams.

The present invention further provides cosmetic, dermatological and/or pharmaceutical formulations comprising the polysiloxanes according to the general formula 1.

Preferred formulations according to the invention are those in which the use described above is preferably carried out.

According to the invention, it is preferred that inventive formulations which comprise fatty alcohol ethoxylates comprise at least one further component which is selected from at least one of the two groups “ionic surfactants” or “fatty alcohols”. Formulations preferred according to the invention comprise the polysiloxane of the general formula 1 in a concentration of from 0.01 to 20 mass percent, preferably 0.1 to 8 mass percent, particularly preferably from 0.2 to 4 mass percent, very particularly preferably from 0.2 to 1.0, in particular up to 0.7, mass percent, based on the total formulation.

The formulations according to the invention are preferably cosmetic skincare and haircare formulations, in particular hair shampoos, conditioners and rinses, which are washed out following application (so-called rinse-off formulations), such as, for example, shampoos and conditioners.

Shampoos preferred according to the invention are characterized in that they comprise, as further component, a deposition polymer, such as, for example, quaternized polysaccharides, quaternized polyacrylates, quaternized polycelluloses, quaternized starches, quaternized guar or other quaternized or aminic polymers from the group of polyquaternium (PQ) compounds (INCI name); in particular, such quats are selected from the group consisting of guar quat (for example Hydroxypropyl Guar Hydroxypropyltrimonium Chloride, Guar hydroxypropyltrimonium Chloride or Guar hydroxypropyltrimonium chloride) or the polyquaternium (PQ) compounds, such as, for example PQ-10, PQ-7, PQ-22, PQ-49, PQ 47, PQ-67 or PQ-6.

Shampoos preferred according to the invention are characterized in that they comprise, as further component, at least one anionic surfactant (for example alkyl ether sulphate, alkyl sulphate or alkylbenzenesulphonate) and at least one further surfactant from the group of alkylbetaines or alkyl oligoglucosides or mono- and/or dialkylsuiphosuccinates or alkylamidobetaines or fatty acid sarcosinates.

Conditioners preferred according to the invention are characterized in that they comprise in particular at least one quaternized or aminic or imidazolium-group-containing organic compound, such as, for example, cetrimonium chloride, dicetyldimonium chloride, quaternium-18, behentrimonium chloride, distearyldimonium chloride, quaternium-87, palmitamidopropyltrimonium chloride and the corresponding methosulphates or the amidoamines stearamidopropyldimethylamine and behenylamidopropyldimethylamine.

The formulation according to the invention can, for example, comprise at least one additional component, selected from the group of

emollients,
emulsifiers,
thickeners/viscosity regulators/stabilizers,
antioxidants,
hydrotropes (or polyols),
solids and fillers,
pearlescence additives,
deodorant and antiperspirant active ingredients,
insect repellents,
self-tanning agent,
preservatives,
conditioners,
perfume,
dyes,
cosmetic active ingredients,
care additives,
superfatting agents,
solvents.

Substances which can be used as exemplary representatives of the individual groups are known to the person skilled in the art and can be found, for example, in the German application DE 102008001788.4. This patent application is hereby incorporated by reference and thus forms part of the disclosure.

As regards further optional components, and also the employed amounts of these components reference is made expressly to the relevant handbooks known to the person skilled in the art, for example K. Schrader, “Grundlagen and Rezepturen der Kosmetika”, [Fundamentals and Formulations of Cosmetics”], 2nd edition, page 329 to 341, Hüthig Buch Verlag Heidelberg.

The amounts of the respective additives are determined according to the intended use. Typical guide formulations for the respective applications are known prior art and are contained, for example, in the brochures of the manufacturers of the particular basic ingredients and active ingredients. These existing formulations can usually be transferred unchanged. If necessary, for adaptation and optimization, the desired modifications, however, can be undertaken without complication by means of simple experiments.

In the examples listed below, the present invention is described by way of example without any intention of limiting the invention, the scope of application of which arises from the overall description and the claims, to the embodiments specified in the examples.

### EXAMPLES

The recording and interpretation of the NMR spectra is known to the person skilled in the art. By way of reference, the book “NMR Spectra of Polymers and Polymer Additives” by A. Brandolini and D. Hills, published in 2000 by Verlag Marcel Dekker Inc., may be introduced herewith.

### Example 1

### Preparation of a Gluconolactampropyl-Diethoxymethylsilane According to Formula 2b

In a 250-ml four-neck flask with attached precision-ground glass stirrer, dropping funnel, reflux condenser and internal thermometer, 35.62 g of D(±)-glucono-β-lactone (99% strength, Sigma Aldrich) are suspended in 35 g of 2-propanol at 70° C. and stirred for 1 hour. At 75° C., 38.62 g of 3-aminopropylmethyldiethoxysilane (Dynasylan® 1505, Evonik Degussa GmbH) are added dropwise over 5 minutes. The mixture is stirred for a further 4 hours at 75° C. This gives a clear, slightly yellowish product with a solids content of 64.8%. The solids content is determined by distilling off the solvent for 2 hours on a rotary evaporator at 60° C. and 20 mbar, and then weighing. The ¹³C-NMR spectrum reveals a complete reaction with the gluconolactone since there are no signals at 45 ppm which would indicate residual amounts of a CH₂—NH₂ group.

### Example 2

### Preparation of an Ethanolic Solution Comprising 3-guanidinopropylmethyldiethoxysilane According to Formula 2c and 3-aminopropylmethyldiethoxysilane

In a 500-ml four-neck flask with attached precision-ground glass stirrer, dropping funnel, reflux condenser and internal thermometer, 95.67 g of 3-aminopropylmethyldiethoxysilane (Dynasylan® 1505, Evonik Degussa GmbH) and 70 g of ethanol are introduced as initial charge. With stirring and at room temperature, 27 g of acetic acid (99-100% strength, J. T. Baker) are added dropwise over 15 minutes. The mixture is heated to 79° C. and, with stirring, 10.51 g of Cyanamid F 1000 (Alzchem Trostberg GmbH), dissolved in 30 g of ethanol, are added dropwise over a period of 2 hours. The mixture is stirred for a further 4 hours at 79° C. This gives a clear, colourless product with a solids content of 54.9%. The ratio, determined by means of ¹³C-NMR, of aminopropylsilane to guanidinopropylsilane is 3:2.

### Example 3

### Preparation of a Gluconolactampropyl- and Aminopropyl-Functional Polysiloxane

In a 500-ml four-neck flask with attached precision-ground glass stirrer, dropping funnel, reflux condenser and internal thermometer, 200 g of dihydroxy-functional polydimethylsiloxane with a chain length of 47.2 dimethylsiloxane units, 6.52 g of 3-aminopropylmethyldiethoxysilane (Dynasylan® 1505, Evonik Degussa GmbH) and 6.48 g of the 64.8% strength 2-propanolic silane solution from example 1 are heated to 85° C. with stirring. 0.68 g of acetic acid (99-100% strength, J. T. Baker) are added and a vacuum is applied. The mixture is stirred for one hour at 85° C. and 20 mbar. The vacuum is broken and, after adding 1.28 g of hexamethyldisilazane (98.5% strength, ABCR GmbH), the mixture is stirred for 1 hour at 85° C. and room pressure. The mixture is then distilled for 1 hour at 85° C. and 20 mbar, 53.57 g of Tegosoft® P (Evonik Goldschmidt GmbH) are added and the mixture is distilled for a further 2 hours. This gives a clear, slightly yellowish product with a viscosity of 320 000 mPa*s at 25° C. The fraction, determined by means of ²⁹Si-NMR, of the chain ends end-capped with trimethylsilyl groups is 65%.

### Example 4

### Preparation of a Guanidinopropyl- and Aminopropyl-Functional Polysiloxane

In a 1000-ml four-neck flask with attached precision-ground glass stirrer, dropping funnel, reflux condenser and internal thermometer, 656.3 g of dihydroxy-functional polydimethylsiloxane with a chain length of 47.6 dimethylsiloxane units, 10.62 g of 3-aminopropylmethyldiethoxysilane (Dynasylan® 1505, Evonik Degussa GmbH) and 26.95 g of the 54.9% strength ethanolic silane solution from example 2 are heated to 85° C. with stirring. The mixture is stirred for one hour at 85° C. and 20 mbar. The vacuum is broken and, after adding 4.18 g of hexamethyldisilazane (98.5% strength, ABCR GmbH), the mixture is stirred for 1 hour at 85° C. and room pressure. The mixture is then distilled for 3 hours at 85° C. and 20 mbar. This gives a cloudy, colourless product of viscosity 41 500 mPa*s at 25° C. The fraction, determined by means of ²⁹Si-NMR of the chain ends end-capped with trimethylsilyl groups is 80%. The potentiometric titration of the product with two different strength basic, nitrogen-containing groups has two transition points.

### Example 5

### Preparation of an Octadecyl-Modified Aminosiloxane

In a 500-ml four-neck flask with attached precision-ground glass stirrer, dropping funnel, reflux condenser and internal thermometer, 246.6 g of dihydroxy-functional polydimethylsiloxane with a chain length of 47.2 dimethylsiloxane units, 9.64 g of 3-aminopropylmethyldiethoxysilane (Dynasylan® 1505, Evonik Degussa GmbH), 2.01 g of octadecylmethyldimethoxysilane (Wacker AG) and 1.18 g of acetic acid (99-100% strength, J. T. Baker) are heated to 85° C. with stirring. The mixture is distilled for one hour at 85° C. and 20 mbar. The vacuum is broken and, after adding 1.28 g of hexamethyldisilazane (98.5% strength, ABCR GmbH), the mixture is stirred for 1 hour at 85° C. and room pressure. The mixture is them distilled for 3 hours at 85° C. and 20 mbar. This gives a colourless, slightly cloudy product with a viscosity of 1520 mPa*s at 25° C. The fraction, determined by means of ²⁹Si-NMR, of the chain ends end-capped with trimethylsilyl groups is 75%.

### Comparison Product 1 and 2

### Comparison Product 1:

Momentive SF 1708 (INCI: Amodimethicone) commercially available from Momentive.
Comparison product 2:
Dow Corning 2-8566 (INCI: Amodimethicone) commercially available from Dow Corning.

Both comparison products are very good conditioners and are used in a large number of cosmetic applications.

### Application Properties

The formulation constituents are named in the compositions in the form of the generally recognized INCI nomenclature. All concentrations in the application examples are given in percent by weight.

### AT1a) Testing the Conditioning of Skin by Means of a Hand Washing Test:

To assess the conditioning of skin (skincare effect) of example No. 3 according to the invention in aqueous, surface-active formulations, sensory hand washing tests were carried out in comparison to comparative example 2 according to the prior art. Comparative example 2 is widespread in the industry as a care active ingredient and serves as a highly effective care active ingredient in aqueous, surface-active formulations.

A group consisting of 10 trained test subjects washed their hands in a defined manner and evaluated foam properties and skin feel by reference to a grading scale from 1 (poor) to 5 (very good).

The products used were in each case tested in a standardized surfactant formulation (Table 1).

The control formulation 0b used is a formulation without the addition of an organomodified siloxane.

TABLE 1
Test formulations for hand washing test.

| | Formulation examples | | |
|---|---|---|---|
| | 0b | 1b | V2b |
| Texapon NSO ®, 28% strength, Cognis | 32% | 32% | 32% |
| (INCI: Sodium Laureth Sulfate) | | | |
| TEGO Betain F 50 ®, 38% strength, | 8% | 8% | 8% |
| Evonik Goldschmidt GmbH | | | |
| (INCI: Cocamidopropyl Betaine) | | | |
| NaCl | 2% | 2% | 2% |
| Water, demineralized | ad 100.0% | | |
| Example 3 (according to the invention) | | 0.5% | |
| Comparative example 2 | | | 0.5% |
| (not according to the invention) | | | |

The sensory test results are summarized in Table 2.

TABLE 2
Results of the hand washing test

| | Test formulation | | |
|---|---|---|---|
| | 0b | 1b | V2b |
| Skin feel during washing | 2.6 | 3.8 | 3.5 |
| Skin smoothness | 1.4 | 3.6 | 2.9 |
| Skin softness | 2.0 | 3.5 | 2.9 |
| Skin smoothness after 3 min | 2.6 | 3.9 | 3.4 |
| Skin softness after 3 min | 2.5 | 3.8 | 3.3 |

Table 2 shows the results of the hand washing test. It is evident from the measurement results that the formulation 1b according to the invention when using example 3 according to the invention is superior in all application properties compared to comparison formulation V2b according to the prior art.

Against this background, the results of formulation 1b according to the invention are to be denoted as very good.

It is evident from the measurement values that example 3 according to the invention in formulation 1b leads to an improvement in the skin properties compared to comparative example 2 in formulation V2b.

In addition, the measurement values reveal that the control formulation 0b without a silicone compound has poorer measurement values than formulations 1b and V2b. ATM) Testing of the conditioning of skin (skincare effect) by means of a hand washing test:

To assess the conditioning of skin (skincare effect) and the foam properties of example 3 according to the invention in aqueous, surface-active formulations, sensory hand washing tests were carried out in comparison to comparative example 2 according to the prior art. This time a lower use concentration was used compared to comparative example 2 in order to test example 3 according to the invention as to higher effectiveness.

The products used were in each case tested in a standardized surfactant formulation (Table 3).

The control formulation 0c used is a formulation without the addition of an organomodified siloxane.

TABLE 3
Test formulations for hand washing test.

| | Formulation examples | | |
|---|---|---|---|
| | 0c | 1c | V2c |
| Texapon NSO ®, 28% strength, Cognis | 32% | 32% | 32% |
| (INCI: Sodium Laureth Sulfate) | | | |
| TEGO Betain F 50 ®, 38% strength, | 8% | 8% | 8% |
| Evonik Goldschmidt GmbH | | | |
| (INCI: Cocamidopropyl Betaine) | | | |
| NaCl | 2% | 2% | 2% |
| Water, demineralized | ad 100.0% | | |
| Example 3 (according to the invention) | | 0.4% | |
| Comparative example 2 | | | 0.5% |
| (not according to the invention) | | | |

The sensory test results are summarized in Table 4.

TABLE 4
Results of the hand washing test

| | Test formulation | | |
|---|---|---|---|
| | 0c | 1c | V2c |
| Skin feel during washing | 2.6 | 3.6 | 3.5 |
| Skin smoothness | 1.4 | 3.2 | 2.9 |
| Skin softness | 2.0 | 3.2 | 2.9 |
| Skin smoothness after 3 min | 2.6 | 3.4 | 3.4 |
| Skin softness after 3 min | 2.5 | 3.4 | 3.3 |

Table 4 shows the results of the hand washing test. It is evident from the measurement results that the formulation 1c according to the invention when using example 3 according to the invention is superior or equivalent in all application properties compared to comparison formulation V2c according to the prior art.

The results show that compound 4 according to the invention has higher effectiveness than comparison compound 2. This shows that example 3 according to the invention has higher substantivity than comparative example 2.

### A T2a) Testing the Conditioning of Hair by Means of Sensory Tests:

For the applications-related assessment of the conditioning of hair, example 4 according to the invention and comparative example 1 were used in simple cosmetic formulations (shampoo and hair rinse).

The application properties upon use in a shampoo were tested in the following formulations:

TABLE 5
Shampoo formulations for testing the hair-conditioning properties.

| | Formulation examples | | |
|---|---|---|---|
| | 0d | 1d | V2d |
| Texapon NSO ®, 28% strength, Cognis | 32% | 32% | 32% |
| (INCI: Sodium Laureth Sulfate) | | | |
| TEGO ® Betain F 50, 38% strength, | 8% | 8% | 8% |
| Evonik Goldschmidt GmbH | | | |
| (INCI: Cocamidopropyl Betaine) | | | |
| Jaguar 162, Rhodia | 0.3% | 0.3% | 0.3% |
| (INCI: Guar Hydroxypropyltrimonium | | | |
| Chloride) (cationic polymer for improving the | | | |
| effectiveness of conditioners) | | | |
| Water, demineralized | ad 100.0% | | |
| Citric acid | ad pH | | |
| | 6.0 ± 0.3 | | |
| Example 4 (according to the invention) | | 0.5% | |
| Comparative example 1 (not according to | | | 0.5% |
| the invention) | | | |

To assess the properties of the shampoo formulation, no after-treatment with a rinse was carried out in the course of the test.

The application properties upon use in hair rinses were tested in the following formulations:

TABLE 6
Hair rinse formulations for testing the hair-conditioning properties.

| | Formulation examples | | |
|---|---|---|---|
| | 0e | 1e | V2e |
| TEGINACID ® C, Evonik Goldschmidt | 0.5% | 0.5% | 0.5% |
| GmbH (INCI: Ceteareth-25) | | | |
| TEGO ® Alkanol 16, Evonik Goldschmidt | 4% | 4% | 4% |
| GmbH (INCI: Cetyl Alcohol) | | | |
| VARISOFT ® 300, 30% strength, Evonik | 3.3% | 3.3% | 3.3% |
| Goldschmidt GmbH | | | |
| (INCI: Cetrimonium Chloride) | | | |
| Water, demineralized | ad 100.0% | | |
| Citric acid | ad. pH | | |
| | 4.0 ± 0.3 | | |
| Example 4 (according to the invention) | | 0.5% | |
| Comparative example 1 (not according to | | | 0.5% |
| the invention) | | | |

In the case of the property testing of hair rinses, the hair is pre-treated by means of a shampoo which does not contain a conditioner.

For the applications-related assessment, hair tresses which are used for sensory tests are pre-damaged by means of a perming treatment and a bleaching treatment in a standardized manner. For this, customary hairstyling products are used. The course of the test, the base materials used and also the details of the assessment criteria are described in DE 103 27 871.

Standardized Treatment of Pre-Damaged Hair Tresses with Conditioning Samples:

The hair tresses, pre-damaged as described above, are treated as follows with the above-described shampoo or the above-described conditioning rinse:

The hair tresses are wetted under running warm water. The excess water is gently squeezed out by hand, then the shampoo is applied and gently worked into the hair (1 ml/hair tress (2 g)). After a time of 1 min, the hair is rinsed for 1 min.

If appropriate, the rinse is applied directly afterwards and gently worked into the hair (1 ml/hair tress (2 g)). After a time of 1 min, the hair is rinsed for 1 min.

Prior to the sensory assessment, the hair is dried in air at 50% humidity and 25° C. for at least 12 h.

### Assessment Criteria:

The sensory evaluations are made according to grades awarded on a scale from 1 to 5, with 1 being the poorest and 5 being the best evaluation. The individual test criteria are in each case given their own assessment.

The test criteria are: wet combability, wet feel, dry combability, dry feel, appearance/shine.

The table below compares the results of the sensory assessment of the treatment of the hair tresses carried out as described above with the formulation 1d according to the invention, the comparison formulation V2d and the control formulation 0d (placebo without test substance).

TABLE 7
Results of the conditioning of hair from shampoo formulation

| | Wet | Wet | Dry | Dry | |
|---|---|---|---|---|---|
| | combability | feel | combability | feel | Shine |
| Formulation 1d | 3.8 | 3.5 | 3.3 | 4.2 | 3.9 |
| according to | | | | | |
| the invention | | | | | |
| Comparison | 3.2 | 3.1 | 3.1 | 3.7 | 3.3 |
| formulation V2d | | | | | |
| (not according to the | | | | | |
| invention) | | | | | |
| Control formulation 0d | 2.3 | 2.5 | 2.5 | 3.3 | 2.3 |
| (placebo) | | | | | |

Surprisingly, the results reveal that formulation 1d according to the invention with example 5 according to the invention is given significantly better evaluations than comparison formulation V2d with comparison example 1 according to the prior art. The good evaluation of the shine properties of all formulations according to the invention is emphasized particularly clearly.

TABLE 8
Results of the conditioning of hair from hair rinse formulations

| | Wet | Wet | Dry | Dry | |
|---|---|---|---|---|---|
| | combability | feel | combability | feel | Shine |
| Formulation 1e | 5.0 | 4.9 | 4.7 | 4.8 | 4.5 |
| according to the | | | | | |
| invention | | | | | |
| Comparison | 4.4 | 4.3 | 4.4 | 4.5 | 3.9 |
| formulation V2e | | | | | |
| (not according to the | | | | | |
| invention) | | | | | |
| Control formulation 0e | 3.8 | 3.9 | 4.0 | 3.8 | 2.9 |

In the application hair rinse too, formulation 1e according to the invention with example 4 according to the invention shows very good cosmetic evaluations in the sensory assessment. In this connection, the already very good properties of comparison formulation V2e with comparative example 1 were yet further increased by formulation 1e according to the invention with example 4 according to the invention.

### A T2b) Testing the Conditioning of Hair by Means of Sensory Tests::

For the applications-related assessment of the conditioning of hair, example 4 according to the invention and comparative example 1 according to the prior art were used in simple cosmetic formulations (shampoo and hair rinse). This time a lower use concentration was used compared to comparative example 1 in order to test example 4 according to the invention as to higher effectiveness. The experiments were carried out analogously to the description see 2a).

The application properties upon use in a shampoo were tested in the following formulations:

TABLE 9
Shampoo formulations for testing the hair-conditioning properties at
various concentrations.

| | Formulation examples | | |
|---|---|---|---|
| | 0f | 1f | V2f |
| Texapon NSO ®, 28% strength, Cognis | 32% | 32% | 32% |
| (INCI: Sodium Laureth Sulfate) | | | |
| TEGO ® Betain F 50, 38% strength, | 8% | 8% | 8% |
| Evonik Goldschmidt GmbH | | | |
| (INCI: Cocamidopropyl Betaine) | | | |
| Jaguar 162, Rhodia | 0.3% | 0.3% | 0.3% |
| (INCI: Guar Hydroxypropyltrimonium | | | |
| Chloride) (cationic polymer for improving the | | | |
| effectiveness of conditioners) | | | |
| Water, demineralized | ad 100.0% | | |
| Citric acid | ad pH | | |
| | 6.0 ± 0.3 | | |
| Example 4 (according to the invention) | | 0.4% | |
| Comparative example 1 (not according | | | 0.5% |
| to the invention) | | | |

The application properties upon use in hair rinses were tested in the following formulations:

TABLE 10
Hair rinse formulations for testing the hair-conditioning properties at
various concentrations.

| | Formulation examples | | |
|---|---|---|---|
| | 0g | 1g | V2g |
| TEGINACID ® C, Evonik Goldschmidt | 0.5% | 0.5% | 0.5% |
| GmbH (INCI: Ceteareth-25) | | | |
| TEGO ® Alkanol 16, Evonik Goldschmidt | 4% | 4% | 4% |
| GmbH (INCI: Cetyl Alcohol) | | | |
| VARISOFT ® 300, 30% strength, Evonik | 3.3% | 3.3% | 3.3% |
| Goldschmidt GmbH | | | |
| (INCI: Cetrimonium Chloride) | | | |
| Water, demineralized | ad 100.0% | | |
| Citric acid | ad pH | | |
| | 4.0 ± 0.3 | | |
| Example 4 (according to the invention) | | 0.4% | |
| Comparative example 1 (not according to the | | | 0.5% |
| invention) | | | |

The table below compares the results of the sensory assessment of the treatment of the hair tresses carried out as described above with formulation 1d according to the invention, comparison formulation V2d and control formulation 0d (placebo without test substance).

TABLE 11
Results of the conditioning of hair from shampoo formulation when using
different concentrations

| | Wet | Wet | Dry | Dry | |
|---|---|---|---|---|---|
| | combability | feel | combability | feel | Shine |
| Formulation 1f | 3.3 | 3.3 | 3.1 | 3.9 | 3.3 |
| according to | | | | | |
| the invention | | | | | |
| Comparison | 3.2 | 3.1 | 3.1 | 3.7 | 3.3 |
| formulation V2f | | | | | |
| (not according to | | | | | |
| the invention) | | | | | |
| Control formulation 0f | 2.3 | 2.5 | 2.5 | 3.3 | 2.3 |
| (placebo) | | | | | |

Surprisingly, the results reveal that formulation if according to the invention with example 4 according to the invention is superior or equivalent in all application properties compared to comparison formulation V2f according to the prior art although 20% less example 4 than comparative example 1 was used in the respective formulations.

The results show that example 4 according to the invention has higher effectiveness than comparative example 1. This shows that example 4 according to the invention has higher substantivity than comparative example 1.

TABLE 12
Results of the conditioning of hair from hair rinse formulations when using
various concentrations

| | Wet | Wet | Dry | Dry | |
|---|---|---|---|---|---|
| | combability | feel | combability | feel | Shine |
| Formulation 1g | 4.5 | 4.4 | 4.4 | 4.5 | 4.1 |
| according to | | | | | |
| the invention | | | | | |
| Comparison | 4.4 | 4.3 | 4.4 | 4.5 | 3.9 |
| formulation V2g | | | | | |
| (not according to the | | | | | |
| invention) | | | | | |
| Control formulation 0g | 3.8 | 3.9 | 4.0 | 3.8 | 2.9 |

In the application hair rinse too, formulation 1g according to the invention with example 4 according to the invention exhibits very good cosmetic evaluations in the sensory assessment. In this connection, the already very good properties of comparison formulation V2g with comparative example 1 were at least achieved or slightly surpassed by formulation 1g according to the invention with example 4 according to the invention although 20% less example 4 than comparative example 1 was used in the respective formulations.

The results show that example 4 according to the invention has higher effectiveness than comparative example 1. This shows that example 4 according to the invention has higher substantivity than comparative example 1.

### Formulation Examples

The following formulation examples show that polysiloxanes according to formula 1 which are modified with lateral amino functions and at least one further lateral functional group in defined ratios can be used in a large number of cosmetic formulations.

### Formulation Example 1) Clear Shampoo

| | |
|---|---|
| TEXAPON ® NSO, Cognis, 28% strength | 32.00% |
| (INCI: Sodium Laureth Sulfate) | |
| Compound example 4 | 0.50% |
| Perfume | 0.50% |
| Water | 57.50% |
| TEGO ® Betain F 50, Evonik Goldschmidt GmbH, 38% | 8.00% |
| strength | |
| (INCI: Cocamidopropyl Betaine) | |
| ANTIL ® 171 Evonik Goldschmidt GmbH | 1.00% |
| (INCI: PEG-18 Glyceryl Oleate/Cocoate) | |
| NaCl | 0.50% |
| Preservative | q.s. |

### Formulation Example 2) Conditioning Shampoo

| | |
|---|---|
| TEXAPON ® NSO, Cognis, 28% strength | 32.00% |
| (INCI: Sodium Laureth Sulfate) | |
| Compound example 4 | 1.00% |
| Perfume | 0.50% |
| Water | 55.70% |
| TEGO ® Cosmo C 100, Evonik Goldschmidt GmbH, | 1.00% |
| (INCI: Creatine) | |
| Jaguar C-162, Rhodia | 0.30% |
| (INCI: Hydroxypropyl Guar Hydroxypropyltrimonium | |
| Chloride) | |
| TEGO ® Betain F 50, Evonik Goldschmidt GmbH, 38% | 8.00% |
| strength | |
| (INCI: Cocamidopropyl Betaine) | |
| NaCl | 1.50% |
| Preservative | q.s. |

### Formulation Example 3) Conditioning Shampoo

| | |
|---|---|
| TEXAPON ® NSO, Cognis, 28% strength | 32.00% |
| (INCI: Sodium Laureth Sulfate) | |
| ANTIL ® 200, Evonik Goldschmidt GmbH (INCI: PEG-200 | 2.00% |
| Hydrogenated Glyceryl Palmate; PEG-7 Glyceryl | |
| Cocoate) | |
| Compound example 2 | 1.00% |
| Perfume | 0.25% |
| Water | 56.25% |
| Polymer JR 400, Amerchol | 0.20% |
| (INCI: Polyquaternium-10) | |
| TEGO ® Betain F 50, Evonik Goldschmidt GmbH, 38% | 8.00% |
| strength | |
| (INCI: Cocamidopropyl Betaine) | |
| NaCl | 0.30% |
| Preservative | q.s. |

### Formulation Example 4) Conditioning Shampoo

| | |
|---|---|
| TEXAPON ® NSO, Cognis, 28% strength | 32.00% |
| (INCI: Sodium Laureth Sulfate) | |
| ANTIL ® 200, Evonik Goldschmidt GmbH (INCI: PEG-200 | 2.00% |
| Hydrogenated Glyceryl Palmate; PEG-7 Glyceryl | |
| Cocoate) | |
| ABIL ® Quat 3272, Evonik Goldschmidt GmbH | 0.75% |
| (INCI: Quaternium-80) | |
| Compound example 1 | 0.50% |
| Perfume | 0.25% |
| Water | 56.00% |
| Polymer JR 400, Amerchol | 0.20% |
| (INCI: Polyquaternium-10) | |
| TEGO ® Betain F 50, Evonik Goldschmidt GmbH, 38% | 8.00% |
| strength | |
| (INCI: Cocamidopropyl Betaine) | |
| NaCl | 0.30% |
| Preservative | q.s. |

### Formulation Example 5) Conditioning Shampoo

| | |
|---|---|
| TEXAPON ® NSO, Cognis, 28% strength | 32.00% |
| (INCI: Sodium Laureth Sulfate) | |
| ANTIL ® 200, Evonik Goldschmidt GmbH (INCI: PEG-200 | 2.00% |
| Hydrogenated Glyceryl Palmate; PEG-7 Glyceryl | |
| Cocoate) | |
| ABIL ® B 8832, Evonik Goldschmidt GmbH | 1.00% |
| (INCI: Bis-PEG/PPG-20/20 Dimethicone) | |
| Compound example 4 | 0.50% |
| Perfume | 0.25% |
| Water | 55.75% |
| Polymer JR 400, Amerchol | 0.20% |
| (INCI: Polyquaternium-10) | |
| TEGO ® Betain F 50, Evonik Goldschmidt GmbH, 38% | 8.00% |
| strength | |
| (INCI: Cocamidopropyl Betaine) | |
| NaCl | 0.30% |
| Preservative | q.s. |

### Formulation Example 6) Conditioning Shampoo

| | |
|---|---|
| TEXAPON ® NSO, Cognis, 28% strength | 32.00% |
| (INCI: Sodium Laureth Sulfate) | |
| VARISOFT ® PATC, Evonik Goldschmidt GmbH | 1.50% |
| (INCI: Palmitamidopropyltrimonium Chloride) | |
| REWODERM ® LI S 80, Evonik Goldschmidt GmbH | 2.00% |
| (INCI: PEG-200 Hydrogenated Glyceryl Palmate; PEG-7 | |
| Glyceryl Cocoate) | |
| Compound example 3 | 0.50% |
| Perfume | 0.25% |
| Water | 54.05% |
| TEGO ® Cosmo C 100, Evonik Goldschmidt GmbH, | 1.00% |
| (INCI: Creatine) | |
| Jaguar C-162, Rhodia | 0.20% |
| (INCI: Hydroxypropyl Guar Hydroxypropyltrimonium | |
| Chloride) | |
| TEGO ® Betain F 50, Evonik Goldschmidt GmbH, 38% | 8.00% |
| strength | |
| (INCI: Cocamidopropyl Betaine) | |
| NaCl | 0.50% |
| Preservative | q.s. |

### Formulation Example 7) Conditioning Shampoo

| | |
|---|---|
| TEXAPON ® NSO, Cognis, 28% strength | 32.00% |
| (INCI: Sodium Laureth Sulfate) | |
| REWODERM ® LI S 80, Evonik Goldschmidt GmbH | 2.00% |
| (INCI: PEG-200 Hydrogenated Glyceryl Palmate; PEG-7 | |
| Glyceryl Cocoate) | |
| Compound example 2 | 0.50% |
| Perfume | 0.25% |
| Water | 55.55% |
| TEGO ® Cosmo C 100, Evonik Goldschmidt GmbH, | 1.00% |
| (INCI: Creatine) | |
| Jaguar C-162, Rhodia | 0.20% |
| (INCI: Hydroxypropyl Guar Hydroxypropyltrimonium | |
| Chloride) | |
| TEGO ® Betain F 50, Evonik Goldschmidt GmbH, 38% | 8.00% |
| strength | |
| (INCI: Cocamidopropyl Betaine) | |
| NaCl | 0.50% |
| Preservative | q.s. |

### Formulation Example 8) Pearlized Shampoo

| | |
|---|---|
| TEXAPON ® NSO, Cognis, 28% strength | 32.00% |
| (INCI: Sodium Laureth Sulfate) | |
| Compound example 4 | 0.50% |
| Perfume | 0.25% |
| Water | 55.25% |
| TEGO ® Betain F 50, Evonik Goldschmidt GmbH, 38% | 8.00% |
| strength | |
| (INCI: Cocamidopropyl Betaine) | |
| TEGO ® Pearl N 300 Evonik Goldschmidt GmbH | 2.00% |
| (INCI: Glycol Distearate; Laureth-4; Cocamidopropyl | |
| Betaine) | |
| ANTIL ® 171 Evonik Goldschmidt GmbH | 1.50% |
| (INCI: PEG-18 Glyceryl Oleate/Cocoate) | |
| NaCl | 0.50% |
| Preservative | q.s. |

Formulation Example 9) 2-in-1 Shampoo

| | |
|---|---|
| TEXAPON ® NSO, Cognis, 28% strength | 32.00% |
| (INCI: Sodium Laureth Sulfate) | |
| VARISOFT ® PATC, Evonik Goldschmidt GmbH | 1.50% |
| (INCI: Palmitamidopropyltrimonium Chloride) | |
| REWODERM ® LI S 80, Evonik Goldschmidt GmbH | 2.00% |
| (INCI: PEG-200 Hydrogenated Glyceryl Palmate; PEG-7 | |
| Glyceryl Cocoate) | |
| Compound example 1 | 0.50% |
| Perfume | 0.25% |
| Water | 54.05% |
| TEGO ® Cosmo C 100, Evonik Goldschmidt GmbH, | 1.00% |
| (INCI: Creatine) | |
| Jaguar C-162, Rhodia | 0.20% |
| (INCI: Hydroxypropyl Guar Hydroxypropyltrimonium | |
| Chloride) | |
| TEGO ® Betain F 50, Evonik Goldschmidt GmbH, 38% | 8.00% |
| strength | |
| (INCI: Cocamidopropyl Betaine) | |
| NaCl | 0.50% |
| Preservative | q.s. |

### Formulation Example 10) Rinse-Off Conditioner

| | |
|---|---|
| Water | 90.50% |
| VARISOFT ® BT 85, Evonik Goldschmidt GmbH | 3.00% |
| (INCI: Behentrimonium Chloride) | |
| Compound example 5 | 1.50% |
| TEGO ® Alkanol 1618, Evonik Goldschmidt GmbH | 5.00% |
| (INCI: Cetearyl Alcohol) | |
| Preservative, Perfume | q.s. |

### Formulation Example 11) Rinse-Off Conditioner

| | |
|---|---|
| Water | 90.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt GmbH | 2.00% |
| (INCI: Behentrimonium Chloride) | |
| Compound example 4 | 0.80% |
| TEGO ® Alkanol 1618, Evonik Goldschmidt GmbH | 5.00% |
| (INCI: Cetearyl Alcohol) | |
| Preservative, Perfume | q.s. |

### Formulation Example 12) Rinse-Off Conditioner

| | |
|---|---|
| Water | 89.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt GmbH | 2.00% |
| (INCI: Behentrimonium Chloride) | |
| ABIL ® Quat 3272, Evonik Goldschmidt GmbH | 1.00% |
| (INCI: Quaternium-80) | |
| Compound example 1 | 0.80% |
| TEGO ® Alkanol 1618, Evonik Goldschmidt GmbH | 5.00% |
| (INCI: Cetearyl Alcohol) | |
| Preservative, Perfume | q.s. |

### Formulation Example 13) Rinse-Off Conditioner

| | |
|---|---|
| TEGINACID ® C, Evonik Goldschmidt GmbH | 0.50% |
| (INCI: Ceteareth-25) | |
| TEGO ® Alkanol 16, Evonik Goldschmidt GmbH | 2.00% |
| (INCI: Cetyl Alcohol) | |
| TEGO ® Amid S 18, Evonik Goldschmidt GmbH | 1.00% |
| (INCI: Stearamidopropyl Dimethylamine) | |
| Compound example 2 | 1.50% |
| Propylene Glycol | 2.00% |
| Citric Acid Monohydrate | 0.30% |
| Water | 92.70% |
| Preservative, Perfume | q.s. |

### Formulation Example 141 Rinse-Off Conditioner

| | |
|---|---|
| TEGINACID ® C, Evonik Goldschmidt GmbH | 0.50% |
| (INCI: Ceteareth-25) | |
| TEGO ® Alkanol 16, Evonik Goldschmidt GmbH | 5.00% |
| (INCI: Cetyl Alcohol) | |
| TEGOSOFT ® DEC, Evonik Goldschmidt GmbH | 1.00% |
| (INCI: Diethylhexyl Carbonate) | |
| Compound example 4 | 1.50% |
| Water | 89.20% |
| TEGO ® Cosmo C 100 Evonik Goldschmidt GmbH | 0.50% |
| (INCI: Creatine) | |
| Propylene Glycol | 2.00% |
| Citric Acid Monohydrate | 0.30% |
| Preservative, Perfume | q.s. |

### Formulation Example 15) Leave-in Conditioner Spray

| | |
|---|---|
| Lactic Acid, 80% | 0.40% |
| Water | 95.60% |
| TEGO ® Amid S 18, Evonik Goldschmidt GmbH | 1.20% |
| (INCI: Stearamidopropyl Dimethylamine) | |
| TEGIN ® G 1100 Pellets, Evonik Goldschmidt GmbH | 0.90% |
| (INCI: Glycol Distearate) | |
| TEGO ® Care PS, Evonik Goldschmidt GmbH | 1.20% |
| (INCI: Methyl Glucose Sesquistearate) | |
| TEGOSOFT ® DEC, Evonik Goldschmidt GmbH | 0.30% |
| (INCI: Diethylhexyl Carbonate) | |
| Compound example 4 | 0.40% |
| Preservative, Perfume | q.s. |

### Formulation Example 16) Leave-in Conditioner Spray

| | |
|---|---|
| TAGAT ® CH-40, Evonik Goldschmidt GmbH | 2.00% |
| (INCI: PEG-40 Hydrogenated Castor Oil) | |
| Ceramide VI, Evonik Goldschmidt GmbH | 0.05% |
| (INCI: Ceramide 6 II) | |
| Perfume | 0.20% |
| Water | 90.95% |
| Compound example 1 | 0.50% |
| LACTIL ® Evonik Goldschmidt GmbH | 2.00% |
| (INCI: Sodium Lactate; Sodium PCA; Glycine; Fructose; | |
| Urea; Niacinamide; Inositol; Sodium benzoate; Lactic | |
| Acid) | |
| TEGO ® Betain F 50 Evonik Goldschmidt GmbH 38% | 2.30% |
| (INCI: Cocamidopropyl Betaine) | |
| Citric Acid (10% in water) | 2.00% |

### Formulation Example 17) Leave-in Conditioner Foam

| | |
|---|---|
| Compound example 1 | 0.50% |
| TAGAT ® CH-40, Evonik Goldschmidt GmbH | 0.50% |
| (INCI: PEG-40 Hydrogenated Castor Oil) | |
| Perfume | 0.30% |
| TEGO ® Betain 810, Evonik Goldschmidt GmbH | 2.00% |
| (INCI: Capryl/Capramidopropyl Betaine) | |
| Water | 94.00% |
| TEGO ® Cosmo C 100, Evonik Goldschmidt GmbH | 0.50% |
| (INCI: Creatine) | |
| TEGOCEL ® HPM 50, Evonik Goldschmidt GmbH | 0.30% |
| (INCI: Hydroxypropyl Methylcellulose) | |
| VARISOFT ® 300, Evonik Goldschmidt GmbH | 1.30% |
| (INCI: Cetrimonium Chloride) | |
| LACTIL ® Evonik Goldschmidt GmbH | 0.50% |
| (INCI: Sodium Lactate; Sodium PCA; Glycine; Fructose; | |
| Urea; Niacinamide; Inositol; Sodium benzoate; Lactic | |
| Acid) | |
| Citric Acid (30% in water) | 0.10% |
| Preservative | q.s. |

### Formulation Example 18) Strong Hold Styling Gel

| | |
|---|---|
| TEGO ® Carbomer 141, Evonik Goldschmidt GmbH | 1.20% |
| (INCI: Carbomer) | |
| Water | 67.00% |
| NaOH, 25% | 2.70% |
| PVP/VA W-735, ISP | 16.00% |
| (INCI: PVP/VA Copolymer) | |
| Compound example 1 | 0.50% |
| Alcohol Denat. | 10.00% |
| TAGAT ® O 2 V, Evonik Goldschmidt GmbH | 2.00% |
| (INCI: PEG-20 Glyceryl Oleate) | |
| Perfume | 0.30% |
| ABIL ® B 88183, Evonik Goldschmidt GmbH | 0.30% |
| (INCI: PEG/PPG-20/6 Dimethicone) | |
| Preservative | q.s. |

### Formulation Example 19) Foaming Body Care Composition

| | |
|---|---|
| TEXAPON ® NSO, Cognis, 28% strength | 14.30% |
| (INCI: Sodium Laureth Sulfate) | |
| Perfume | 0.30% |
| Compound example 4 | 0.50% |
| REWOTERIC ® AM C, Evonik Goldschmidt GmbH, 32% | 8.00% |
| strength | |
| (INCI: Sodium Cocoamphoacetate) | |
| Water | 74.90% |
| TEGOCEL ® HPM 50, Evonik Goldschmidt GmbH | 0.50% |
| (INCI: Hydroxypropyl Methylcellulose) | |
| LACTIL ®, Evonik Goldschmidt GmbH | 1.00% |
| (INCI: Sodium Lactate; Sodium PCA; Glycine; Fructose; | |
| Urea; Niacinamide; Inositol; Sodium benzoate; Lactic | |
| Acid) | |
| Citric Acid Monohydrate | 0.50% |

### Formulation Example 20) Body Care Composition

| | |
|---|---|
| TEXAPON ® NSO, Cognis, 28% strength | 30.00% |
| (INCI: Sodium Laureth Sulfate) | |
| TEGOSOFT ® PC 31, Evonik Goldschmidt GmbH | 0.50% |
| (INCI: Polyglyceryl-3 Caprate) | |
| Compound example 4 | 0.50% |
| Perfume | 0.30% |
| Water | 53.90% |
| TEGOCEL ® HPM 4000, Evonik Goldschmidt GmbH | 0.30% |
| (INCI: Hydroxypropyl Methylcellulose) | |
| REWOTERIC ® AM C, Evonik Goldschmidt GmbH, 32% | 10.00% |
| strength | |
| (INCI: Sodium Cocoamphoacetate) | |
| Citric Acid Monohydrate | 0.50% |
| REWODERM ® LI S 80, Evonik Goldschmidt GmbH | 2.00% |
| (INCI: PEG-200 Hydrogenated Glyceryl Palmate; PEG-7 | |
| Glyceryl Cocoate) | |
| TEGO ® Pearl N 300, Evonik Goldschmidt GmbH | 2.00% |
| (INCI: Glycol Distearate; Laureth-4; Cocamidopropyl | |
| Betaine) | |

### Formulation Example 21) Foaming Body Care Composition

| | |
|---|---|
| TEXAPON ® NSO, Cognis, 28% strength | 14.30% |
| (INCI: Sodium Laureth Sulfate) | |
| Perfume | 0.30% |
| Compound example 3 | 0.50% |
| REWOTERIC ® AM C, Evonik Goldschmidt GmbH, 32% | 8.00% |
| strength | |
| (INCI: Sodium Cocoamphoacetate) | |
| Water | 75.10% |
| Polyquaternium-7 | 0.30% |
| LACTIL ®, Evonik Goldschmidt GmbH | 1.00% |
| (INCI: Sodium Lactate; Sodium PCA; Glycine; Fructose; | |
| Urea; Niacinamide; Inositol; Sodium benzoate; Lactic | |
| Acid) | |
| Citric Acid Monohydrate | 0.50% |

### Formulation Example 22) Mild Foam Bath

| | |
|---|---|
| TEXAPON ® NSO, Cognis, 28% strength | 27.00% |
| (INCI: Sodium Laureth Sulfate) | |
| REWOPOL ® SB FA 30, Evonik Goldschmidt GmbH, 40% | 12.00% |
| strength | |
| (INCI: Disodium Laureth Sulfosuccinate) | |
| TEGOSOFT ® LSE 65 K SOFT, Evonik Goldschmidt | 2.00% |
| GmbH | |
| (INCI: Sucrose Cocoate) | |
| Water | 39.00% |
| REWOTERIC ® AM C, Evonik Goldschmidt GmbH, 32% | 13.00% |
| strength | |
| (INCI: Sodium Cocoamphoacetate) | |
| Compound example 2 | 0.50% |
| Citric Acid (30% in water) | 3.00% |
| ANTIL ® 171 Evonik Goldschmidt GmbH | 1.50% |
| (INCI: PEG-18 Glyceryl Oleate/Cocoate) | |
| TEGO ® Pearl N 300 Evonik Goldschmidt GmbH | 2.00% |
| (INCI: Glycol Distearate; Laureth-4; Cocamidopropyl | |
| Betaine) | |

### Formulation Example 23) Foaming Body Care Composition

| | |
|---|---|
| TEGOCEL ® HPM 50, Evonik Goldschmidt GmbH | 0.50% |
| (INCI: Hydroxypropyl Methylcellulose) | |
| Water | 80.10% |
| Perfume | 0.20% |
| Compound example 1 | 0.50% |
| TEGOSOFT ® GC, Evonik Goldschmidt GmbH, | 1.30% |
| (INCI: PEG-7 Glyceryl Cocoate) | |
| TEGO ® Betain 810, Evonik Goldschmidt GmbH | 16.90% |
| (INCI: Capryl/Capramidopropyl Betaine) | |
| LACTIL ®, Evonik Goldschmidt GmbH | 0.50% |
| (INCI: Sodium Lactate; Sodium PCA; Glycine; Fructose; | |
| Urea; Niacinamide; Inositol; Sodium benzoate; Lactic | |
| Acid) | |
| Preservative | q.s. |

### Formulation Example 24) Rinse-Off Conditioner

| | |
|---|---|
| Water | 89.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt GmbH | 2.00% |
| (INCI: Behentrimonium Chloride) | |
| ABIL ® OSW 5, Evonik Goldschmidt GmbH | 1.00% |
| (INCI: Cyclopentasiloxane; Dimethiconol) | |
| Compound example 1 | 0.80% |
| TEGO ® Alkanol 1618, Evonik Goldschmidt GmbH | 5.00% |
| (INCI: Cetearyl Alcohol) | |
| Preservative, Perfume | q.s. |

### Formulation Example 25) Rinse-Off Conditioner

| | |
|---|---|
| Water | 89.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt GmbH | 2.00% |
| (INCI: Behentrimonium Chloride) | |
| ABIL ® Soft AF 100, Evonik Goldschmidt GmbH | 1.00% |
| (INCI: Methoxy PEG/PPG-7/3 Aminopropyl | |
| Dimethicone) | |
| Compound example 1 | 0.80% |
| TEGO ® Alkanol 1618, Evonik Goldschmidt GmbH | 5.00% |
| (INCI: Cetearyl Alcohol) | |
| Preservative, Perfume | q.s. |

### Formulation Example 26) Rinse-Off Conditioner

| | |
|---|---|
| Water | 89.20% |
| VARISOFT ® EQ 65, Evonik Goldschmidt GmbH | 2.00% |
| (INCI: Distearyl Dimonium Chloride, Cetearyl Alcohol) | |
| VARISOFT ® BT 85, Evonik Goldschmidt GmbH | 2.00% |
| (INCI: Behentrimonium Chloride) | |
| SF 1708, Momentive | 1.00% |
| (INCI: Amodimethicone) | |
| Compound example 1 | 0.80% |
| TEGO ® Alkanol 1618, Evonik Goldschmidt GmbH | 5.00% |
| (INCI: Cetearyl Alcohol) | |
| Preservative, Perfume | q.s. |

### Formulation Example 27) Conditioning Shampoo

| | |
|---|---|
| TEXAPON ® NSO, Cognis, 28% strength | 27.00% |
| (INCI: Sodium Laureth Sulfate) | |
| Plantacare 818 UP, Cognis 51.4% strength | 5.00% |
| (INCI: Coco Glucoside) | |
| Compound example 2 | 1.50% |
| Perfume | 0.25% |
| Water | 56.55% |
| TEGO ® Cosmo C 100, Evonik Goldschmidt GmbH, | 1.00% |
| (INCI: Creatine) | |
| Jaguar C-162, Rhodia | 0.20% |
| (INCI: Hydroxypropyl Guar Hydroxypropyltrimonium | |
| Chloride) | |
| TEGO ® Betain F 50, Evonik Goldschmidt GmbH, 38% | 8.00% |
| strength | |
| (INCI: Cocamidopropyl Betaine) | |
| NaCl | 0.50% |
| Preservative | q.s. |

### Formulation Example 28) Conditioning Shampoo

| | |
|---|---|
| Plantacare 818 UP, Cognis 51.4% strength | 18.00% |
| (INCI: Coco Glucoside) | |
| Compound example 2 | 1.50% |
| Perfume | 0.25% |
| Water | 70.55% |
| TEGO ® Cosmo C 100, Evonik Goldschmidt GmbH, | 1.00% |
| (INCI: Creatine) | |
| Jaguar C-162, Rhodia | 0.20% |
| (INCI: Hydroxypropyl Guar Hydroxypropyltrimonium | |
| Chloride) | |
| TEGO ® Betain F 50, Evonik Goldschmidt GmbH, 38% | 8.00% |
| strength | |
| (INCI: Cocamidopropyl Betaine) | |
| NaCl | 0.50% |
| Preservative | q.s. |

### Formulation Example 29) Anti-Ageing Day Cream

| | |
|---|---|
| ABIL ® Care XL 80, Evonik Goldschmidt GmbH, | 1.50% |
| (INCI: Bis-PEG/PPG-20/5 PEG/PPG-20/5 | |
| Dimethicone; Methoxy PEG/PPG-25/4 Dimethicone; | |
| Caprylic/Capric Triglyceride) | |
| Compound example 2 | 1.00% |
| Ceteareth-25 | 1.00% |
| Stearyl Alcohol | 1.50% |
| Glyceryl Stearate | 3.00% |
| Stearic Acid | 1.50% |
| Myristyl Myristate | 1.00% |
| Ceramide IIIB | 0.10% |
| Caprylic/Capric Triglyceride | 5.00% |
| Ethylhexyl Palmitate | 4.40% |
| Ethylhexyl Methoxycinnamate | 2.00% |
| Butyl Methoxydibenzoyl-methane | 1.00% |
| Glycerol | 3.00% |
| Water | ad 100% |
| TEGO ® Carbomer 134, Evonik Goldschmidt GmbH, | 0.10% |
| (INCI: Carbomer) | |
| Ethylhexyl Palmitate | 0.40% |
| Sodium Hydroxide (10% in water) | q.s. |
| Preservative | q.s. |
| Perfume | q.s. |

### Formulation Example 30: Cationic Sun Screen Cream

| | |
|---|---|
| ABIL ® Care XL 80, Evonik Goldschmidt GmbH, | 1.50% |
| (INCI: Bis-PEG/PPG-20/5 PEG/PPG-20/5 | |
| Dimethicone; Methoxy PEG/PPG-25/4 Dimethicone; | |
| Caprylic/Capric Triglyceride) | |
| Compound example 1 | 1.00% |
| Distearyldimonium Chloride | 1.50% |
| Glyceryl Stearate | 2.00% |
| Stearyl Alcohol | 1.00% |
| C12-15 Alkyl Benzoate | 5.00% |
| TEGO ® Sun TDEC 45, Evonik Goldschmidt GmbH, | 5.00% |
| (INCI: Titanium Dioxide; Diethylhexyl Carbonate; | |
| Polyglyceryl-6 Polyhydroxy-stearate) | |
| Diethylhexyl Carbonate | 3.50% |
| Cetyl Ricinoleate | 1.00% |
| Triisostearin | 1.00% |
| Octocrylene | 3.00% |
| Ethylhexyl Methoxycinnamate | 4.00% |
| Butyl Methoxydibenzoylmethane | 2.00% |
| Water | ad 100% |
| Glycerol | 3.00% |
| Preservative | q.s. |
| Perfume | q.s. |

### Formulation Example 31: Self-Tanning Lotion:

| | |
|---|---|
| Polyglyceryl-3 Dicitrate/Stearate | 3.0% |
| Ceteareth-25 | 0.5% |
| Compound example 1 | 0.5% |
| Glyceryl Stearate | 2.5% |
| Stearyl Alcohol | 1.0% |
| Isopropyl Palmitate | 3.0% |
| Caprylic/Capric Triglyceride | 3.0% |
| Mineral Oil | 7.0% |
| Jojoba Oil | 3.0% |
| Glycerol | 3.0% |
| Dihydroxyacetone | 5.0% |
| Demineralized Water | ad 100% |
| Citric Acid (10% solution) (pH adjustment to 4.0) | q.s. |
| Methylisothiazolinone, Methylparaben, Ethylparaben; | 0.8% |
| Dipropylene Glycol | |

### Formulation Example 32: W/O Make-Up

| | |
|---|---|
| Cetyl PEG/PPG-10/1 Dimethicone | 4.5% |
| Cetyl Dimethicone | 1.0% |
| Diethylhexyl Carbonate | 4.5% |
| Ethylhexyl Palmitate | 1.5% |
| Dimethicone | 5.0% |
| Compound example 1 | 0.5% |
| Cyclopentasiloxane | 9.0% |
| Phenyl Trimethicone | 1.0% |
| Lauryl Dimethicone/Polyglyceryl-3 Crosspolymer; | 2.0% |
| Triethylhexanoin*⁾ | |
| Nylon-12 | 1.0% |
| Iron Oxides | 2.0% |
| Titanium Oxide | 6.0% |
| Zinc Oxide | 0.5% |
| Glycerol | 3.0% |
| Sodium Chloride | 0.8% |
| Creatine | 0.2% |
| Water | ad 100% |
| Preservative, Perfume | q.s. |

*⁾KSG-830 (Shin Etsu)

1. A polysiloxane composition of the general formula:
MₐD_{b}D^{A}_{c}D^{B}_{d}D^{e}ₑT_{f}Q_{g} (1)
M=[R²R¹₂SiO_{1/2}]
D=[R¹₂SiO_{2/2}]
D^{A}=[R¹Si(CH₂CH₂CH₂NHR³)O_{2/2}]
D^{B}=[R¹SiR⁴O_{2/2}]
D^{C}=[R¹SiR⁵⁰_{2/2}]
T=[R¹SiO_{3/2}]
Q=[SiO_{4/2}],
wherein:
R¹ is selected from the group consisting of linear or branched, saturated or unsaturated hydrocarbon groups having 1 to 30 carbon atoms, and aromatic hydrocarbon groups having 6 to 30 carbon atoms;
R² is selected from the group consisting of R¹, alkoxy groups, and hydroxy groups;
R³ is selected from the group consisting of hydrogen and hydrocarbon groups substituted with nitrogen atoms;
R⁴ is selected from the group consisting of linear or branched, saturated or olefinically unsaturated hydrocarbon groups having 8 to 30 carbon atoms;
R⁵ is selected from the group consisting of linear or branched, saturated or unsaturated polar hydroxy-substituted amide groups having 1 to 30 carbon atoms; hydroxy-substituted carbamate groups having 1 to 30 carbon atoms; ethoxylated amine groups having 1 to 30 carbon atoms; and guanidine or alkylenylguanidine groups having 1 to 30 carbon atoms;
a=2 to 20
b=10 to 5000
c=1 to 500
d=0 to 500
e=0 to 500
f=0 to 20
g=0 to 20
and ionic adducts thereof with protic reactants H⁺A⁻,
with the proviso that at least 50% of the radicals R²═R¹, and at least one of the indices d and e≠0, and if d=0, then e≠0, and if e=0, then d≠0.

2. The composition of claim 1, wherein R⁵ is selected from: 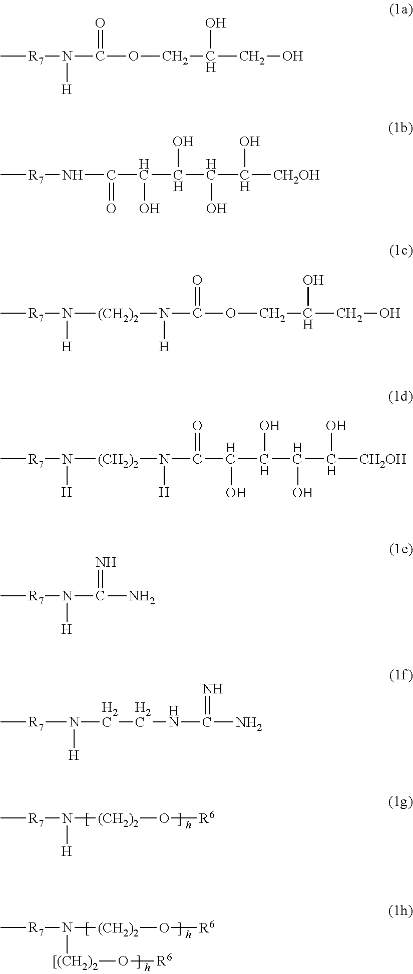
wherein:
R⁶ is selected from the group consisting of hydrogen atom, hydrocarbon groups, acyl groups, carboxylate groups, carbamate groups, and carbonate groups;
R⁷ is selected from the group consisting of linear or branched, saturated or unsaturated, divalent hydrocarbon groups; and
h=0 to 20;
with the proviso that the index c in formula 1 is greater than the index e.

3. The composition of claim 1, wherein R¹ and R² are independently from alkyl radicals having 1 to 4 carbon atoms.

4. The composition of claim 1, further comprising a surfactant.

5.-6. (canceled)

7. A cosmetic, dermatological, or pharmaceutical formulation comprising a polysiloxane according to claim 1.

8. The formulation of claim 7, wherein said polysiloxane is present in a concentration of from 0.01 to 20 mass percent based on the total formulation.

9. (canceled)

10. The formulation of claim 7, wherein the formulation further comprises at least one primary surfactant and at least one further surfactant selected from the group consisting of alkyl oligoglucosides, mono-dialkyl sulfosuccinates, dialkyl sulfosuccinates, alkylamidobetaines and fatty acid sarcosinates.

11. The formulation of claim 7, wherein the formulation further comprises at least one quaternized or aminic or imidazolium-group-containing organic compound.

12. The composition of claim 1, wherein R¹ is a methyl or phenyl group.

13. The composition of claim 1, wherein R² is selected from the group consisting of linear or branched, saturated or unsaturated hydrocarbon groups having 1 to 30 carbon atoms, and aromatic hydrocarbon groups having 6 to 30 carbon atoms.

14. The composition of claim 1, wherein R¹ and R² are methyl.

15. The composition of claim 1, wherein R³ is a hydrogen atom or aminoethyl radical.

16. The composition of claim 1, wherein R⁴ is selected from the group consisting of decyl, dodecyl, tetradecyl, hexadecyl, and octadecyl groups.

17. The composition of claim 1, wherein a is 2 to 10, b is 20 to 2000, c is 1 to 100, d is 0 to 100, e is 1 to 100, f is 0 to 10, and g is 0 to 10.

18. The composition of claim 1, wherein a is 2, b is 20 to 1000, c is 1 to 30, d is 0 to 30, e is 1 to 30, f is 0, and g is 0.

19. The composition of claim 1, wherein at least 70% of the radicals R²═R¹.

20. The composition of claim 2, wherein h is 1 to 10.
